(19) United States
(12) Patent Application Publication
(10) Pub. No.: US 2022/0142753 A1
(43) Pub. Date: May 12, 2022

(54) DENTURE PREPARATION ASSEMBLY, METHOD AND APPARATUS

(71) Applicant: Implant Solutions Pty Ltd, Victoria (AU)

(72) Inventor: Michael Tuckman, Mulgrave, Victoria (AU)

(21) Appl. No.: 17/433,457

(22) PCT Filed: Mar. 6, 2020
(86) PCT No.: PCT/AU2020/050215

§ 371 Date: Aug. 24, 2021

(30) Foreign Application Priority Data
Mar. 7, 2019 (AU) 2019900752

(51) Int. Cl. A61C 13/00; A61C 9/00; A61C 13/34; A61C 13/12; A61C 13/08; A61C 13/01

(52) U.S. Cl. CPC A61C 13/0019; A61C 9/0006; A61C 13/01; A61C 13/12; A61C 13/08; A61C 13/34

(57) ABSTRACT

The invention concerns an apparatus for a denture preparation assembly, comprising an impression tray piece for insertion into the mouth of the patient and to receive impression material and a dental model jig attachable to the impression tray piece, said dental model jig including model teeth, wherein the attachment between the impression tray piece and the dental model jig is adjustable to allow the dental model jig to be moved in the mouth of the patient to a desired position relative to the impression tray piece, said relative position being retainable to allow scanning of the impression material and the dental model jig.

The invention assists in 3D scanning for digital design/manufacture of dentures, and allows the model teeth to be positioned and orientated in the patient's mouth relative to the impression material in the impression tray piece in the patient's mouth, the position and orientation being retained when the apparatus is removed.

### FIELD OF THE INVENTION

The present invention relates to a denture preparation assembly, method and apparatus. In particular, the present invention relates to the digital design and/or manufacture of dentures by scanning impressions taken from a patient.

### BACKGROUND OF THE INVENTION

Dentures are constructed to replace missing teeth for patients who are partially or fully edentulous. Designing and manufacturing a set of dentures which are comfortable when inserted into the mouth of a patient is a complex task. Impressions of edentulous patients must be sufficiently accurate to capture all the details of the patient's oral tissue and mouth. Any imperfections that arise from the impression increase the difficulties of accurately designing the dentures themselves and subsequently affect the production and fit of the dentures.

Trends in the current industry are towards digitally produced dentures, through scanning the impressions taken from a patient, designing a set of dentures in computer software such as CAD, and then manufacturing from the CAD the dentures with the use of CAM software and machining. From the initial set of manufactured dentures, it is common to test a copy of these dentures on the patient and then iteratively modify the dentures in fitting sessions until the final desired set of dentures is produced.

The entire process of constructing dentures using conventional methods and devices therefore generally requires a number of appointments between the dentist and the patient, and involves a significant amount of time and skill. Generally, a patient must make a number of visits to a dental specialist, as many as 4 or 5 visits. Such visits are necessary in order for a dentist to take an impression of the patient's gums, to check the fitting, to design the teeth portions, and then to verify features such as bite registration in the patient's mouth for adjustment.

One known method and apparatus for preparing dentures for a patient is disclosed in U.S. Pat. No. 8,998,615 to Dentca, Inc. However, the general state of the art would be receptive to alternative denture preparation assemblies, methods and apparatuses used in the design and/or manufacture of dentures.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In a first aspect, the present invention provides an apparatus for a denture preparation assembly, said apparatus configured for scanning to facilitate the digital design and/or manufacture of dentures for a patient, the apparatus comprising; an impression tray piece for insertion into the mouth of the patient and to receive impression material; and a dental model jig attachable to the impression tray piece, said dental model jig including model teeth; wherein the attachment between the impression tray piece and the dental model jig is adjustable to allow the dental model jig to be moved in the mouth of the patient to a desired position relative to the impression tray piece, said relative position being retainable to allow scanning of the impression material and the dental model jig.

Preferably, the attachment between the impression tray piece and the dental model jig can be selectively locked to fix the relative position therebetween.

In a preferred form, the dental model jig is slideably attached to the impression tray piece to allow movement in the anterior-posterior direction, the apparatus including a mechanical locking element to selectively block said movement.

Further, the attachment of the dental model jig to the impression tray piece may be configured for adjustment in other directions. This may be achieved by including a connecting portion of a plastically deformable material, such as a length of ductile wire. Preferably, the connecting portion connects the dental model jig to a support portion receivable by the impression tray piece. The support portion may be selectively removably attachable to the impression tray piece in a fixed position. The support portion may include a screw element engageable with a threaded element for locking the dental model jig in position relative to the impression tray piece.

The invention thus allows the model teeth to be positioned and orientated in the patient's mouth relative to the impression material in the impression tray piece while the impression material is engaged over the patient's gum, this position and orientation being retained when the apparatus is removed from the patient's mouth. 3D scanning of the impression material and the dental model jig then provides an accurate digital model for the design and/or manufacture of a denture.

Said model teeth may comprise a plurality of model teeth provided on the dental model jig to mimic real teeth in appearance, or may comprise a single or complex form to model one or more structural or functional characteristics of a set of teeth. Preferably, the apparatus is used for a maxillary denture and said model teeth emulate the central upper front teeth, such as the six central upper front teeth.

Preferably, the impression tray piece is part of a multi-part impression tray having a detachable anterior tray portion, detachment of the anterior tray portion allowing said attachment of the dental model jig.

In this way, the impression tray can be loaded with impression material to take a gum impression including the anterior gum portion, before the anterior tray portion is detached and the impression tray returned to the patient's mouth to locate over the patient's gum for positioning of the dental model jig.

In a second aspect, the present invention provides a denture preparation kit including one or more of the following: a maxillary impression tray; a mandibular impression tray; and an apparatus for a denture preparation assembly in accordance with the first aspect, wherein the dental model jig can be chosen from a plurality of different sized dental model jigs in accordance with the size of the patient's mouth.

Said kit preferably includes an adjustable intra-jaw spacer for use in recording the intra-jaw spacing of upper and lower dentures.

Said kit may further include a handle configured to engage with one or more of the maxillary impression tray and the mandibular impression tray.

In a third aspect, the present invention provides a method for use in denture preparation for a patient, the method comprising: loading an impression material into a first lower impression tray and inserting said lower impression tray into the patient's mouth to take a lower gum impression; removing the first lower impression tray from the patient's mouth; loading an impression material into a second upper impression tray and inserting said second upper impression tray into the patient's mouth to take an upper gum impression; removing the second upper impression tray from the patient's mouth; attaching a dental model jig to the second upper impression tray, said dental model jig including model teeth; re-inserting the second upper impression tray into the patient's mouth over the upper gum; adjusting the dental model jig relative to the second upper impression tray to a desired position, removing the second upper impression tray and dental model jig.

The first lower impression tray and the second upper impression tray with the attached dental model jig are provided for scanning of the lower gum impression, the upper gum impression and the dental model jig (such as scanning of the model teeth) in order for design and/or manufacture of a denture set.

Advantageously, the method allows the modelling of all elements of a denture set in a single visit to a dental clinic, as no further visits should be needed to establish tooth position after the denture gingival portions have been established.

Preferably, the method includes the step of, after the lower impression tray has been removed from the patient's mouth, loading an impression material into a second lower impression tray, the second lower impression tray including an upwardly projecting adjustable intra-jaw spacer, inserting the second lower impression tray into the patient's mouth, and adjusting the intra-jaw spacer.

Preferably, the method further includes the step of, before the second upper impression tray has been inserted into the patient's mouth, loading an impression material into a first upper impression tray, inserting the first lower impression tray into the patient's mouth to take an upper gum impression, and removing the first upper impression tray.

The first and the second upper impression trays may be separate impression trays, or the first upper impression tray may comprise multiple parts, including the second upper impression tray and a removable anterior tray portion. In this case, once the first upper impression tray has been removed from the patient's mouth, the impression material is cut and the removable anterior tray portion removed to allow attachment of said dental model jig. This is then returned to the patient's mouth as the second upper impression tray.

The second upper impression tray may include a downward-facing bearing surface against which the intra-jaw spacer bears in the method including the step of having the intra-jaw spacer bear against said bearing surface in setting a desired spacing between the second upper impression tray and the second lower impression tray.

The method may include the step of introducing a squash bite material between the second lower impression tray and the second upper impression tray and taking a squash bite impression, the squash bite impression defined in part by the spacing set by the intra-jaw spacer.

The method may include the step of scanning the impressions provided by the upper and lower impression trays and the attached dental model jig for use in digital design and/or manufacture of a denture set.

The present invention extends to a method of denture manufacture, including receiving digital data provided by scanning the impressions provided by the upper and lower impression trays and the attached dental model jig resulting from carrying out the above defined method, and manufacturing a denture set in accordance with the digital data.

The manufacture can be carried out by a digital milling machine or a digital 3D printer, for example.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1 provides an inverted exploded perspective view of an apparatus in accordance with an embodiment of the invention;

FIG. 2A provides an upper perspective view of a maxillary impression tray in accordance with an embodiment of the invention;

FIG. 2B provides an inverted perspective view of FIG. 2A;

FIG. 3A provides an inverted perspective view of a dental model receiver in accordance with an embodiment of the invention;

FIG. 3B provides an upper perspective view of the dental model receiver of FIG. 3A with a fixation disc in accordance with an embodiment of the invention;

FIG. 4A provides an upper perspective view of a pin pad and pin pad receiver in accordance with an embodiment of the invention;

FIG. 4B provides an inverted perspective view of the pin pad and pin pad receiver of FIG. 4A connected to a posterior portion of the maxillary impression tray of FIG. 1 in accordance with an embodiment of the invention;

FIG. 5 provides an inverted perspective view of a dental model jig in accordance with an embodiment of the invention;

FIG. 6 illustrates a method in accordance with an embodiment of the invention;

FIG. 7 provides an upper perspective view of a mandibular impression tray in accordance with an embodiment of the invention;

FIG. 8 provides a rear upper perspective view of a bite tray in accordance with an embodiment of the invention;

FIG. 9A provides an upper perspective view of a posterior portion of a maxillary impression tray in accordance with an embodiment of the invention;

FIG. 9B provides an inverted perspective view of an anterior portion of a maxillary impression tray in accordance with an embodiment of the invention;

FIG. 10A provides an upper perspective view of the assembled apparatus of FIG. 1;

FIG. 10B provides an inverted perspective view of FIG. 10A;

FIG. 11 provides an lower perspective view of a posterior portion of a maxillary impression tray in accordance with another embodiment of the invention;

FIG. 12 provides an upper perspective view of the posterior portion of FIG. 11;

FIG. 13 provides an upper perspective view of an anterior portion of a maxillary impression tray in accordance with another embodiment of the invention;

FIG. 14 provides a rear view of the anterior portion of FIG. 13;

FIG. 15 provides an upper perspective view of a dental model jig in accordance with another embodiment of the invention;

FIG. 16 provides a upper perspective view of a bite tray in accordance with an embodiment of the invention; and

FIG. 17 provides a perspective view of a handle in accordance with an embodiment of the invention.

### DETAILED DESCRIPTION OF THE EMBODIMENTS

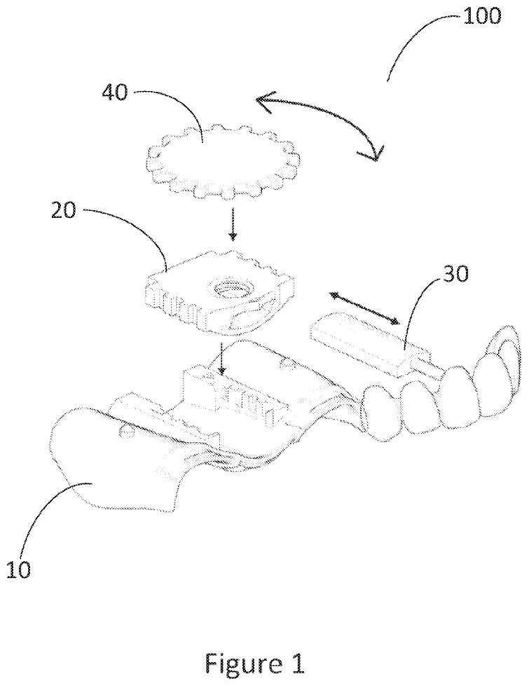

Reference is now made to FIG. 1, which illustrates components of apparatus 100. The apparatus 100 includes an impression tray 10, dental model jig receiver 20, dental model jig 30, and a fixation screw disc 40. Impression tray 10 provides a partial upper impression tray (maxillary tray), which is inserted into the patient's mouth and positioned at the patient's maxillary arch. Hence, it will be appreciated that the apparatus 100 as shown in FIG. 1 is in an inverted view. References herein to a transverse direction are to be taken as references to a direction parallel to a transverse plane as generally understood in the anatomical position (i.e. patient upright). References to a vertical direction refer to the vertical direction as generally understood in the anatomical position. Thus references to upper and lower are to be generally understood based on the positioning of the apparatus 100 (or any other component) with the patient in the upright position.

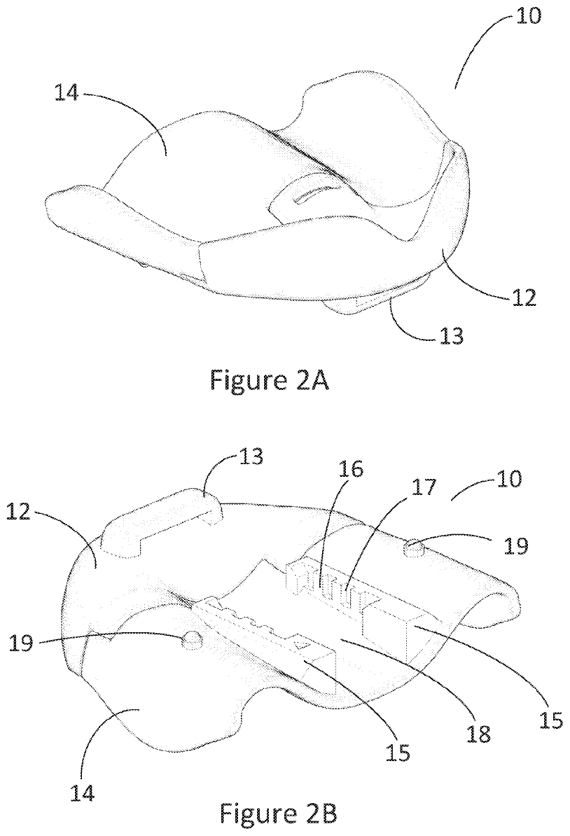

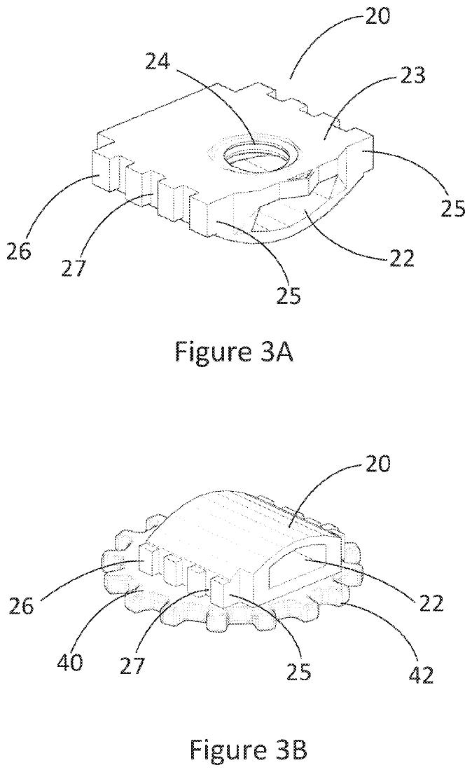

FIGS. 2A and 2B illustrate a two-part maxillary impression tray 10, which when filled with suitable impression material, e.g. alginate impression material, PVS impression material, etc., and inserted into the patient's mouth against the maxillary arch, provides a negative impression of the soft tissues and maxillary arch of the patient's mouth. The maxillary impression tray 10 comprises an anterior portion 12 and posterior portion 14. The anterior portion 12 is firmly but releasably connected to the posterior portion 14 in any suitable removable manner, such as by a snap-fit connection. The anterior portion 12 includes a bracket 13 protruding from a lower surface thereof that allows the maxillary impression tray 10 to be gripped by a suitable dental implement for easy removal of the maxillary tray 10 from the patient's mouth. The posterior portion 14 includes a coupling portion 18 on a lower surface thereof. The coupling portion 18 includes a pair of raised walls 15 protruding vertically downward from the lower surface of the posterior portion 14 and extending substantially from a posterior end to an anterior end of the posterior portion 14. The raised walls 15 include a plurality of spaced fingers 16 and recesses 17 configured for securely receiving the dental model jig receiver 20 or pin pad connector 52 (FIG. 3A, 4A), which both include corresponding shaping for secure mating connection with the posterior portion 14. Once the dental model jig receiver 20 or pin pad connector 52 is coupled to the posterior portion 14, the dental model jig receiver 20 or pin pad connector 52 will be in a fixed position, i.e. not able to translate or rotate relative to the posterior portion 14. Also protruding from the lower surface of the posterior portion 14 is a pair of positioning bosses 19 as shown in FIG. 2B. The function of the positioning bosses 19 will be described later.

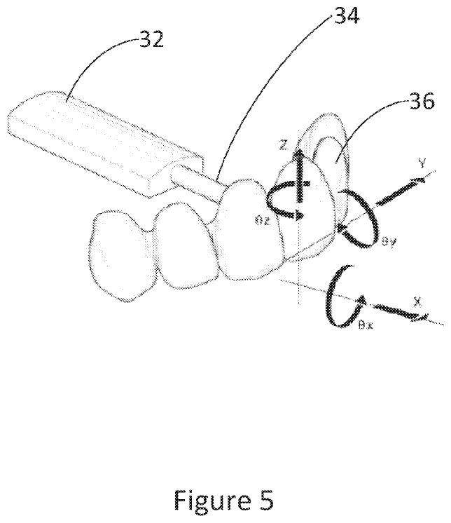

FIGS. 3A and 3B illustrate the dental model receiver 20 having a body 23 substantially in the form of a rectangular prism with a rounded upper facet, to seat against the medial surface of coupling portion 18. The anterior end of the body 23 includes a through cavity 22, through which a slider support body 32 (FIG. 5) of the dental model jig 30 can be received in an anterior-posterior direction. The planar base of body 23 includes a threaded aperture 24 configured to receive a fastening element, namely a grub screw (not shown) projecting from the centre of the upper face of fixation screw disc 40. The grub screw is thus a part of fixation screw disc 40, whereby in use, upon manual rotation of the fixation screw disc 40 in the clockwise direction, the grub screw is threaded into the aperture 24 until it bears onto the slider support body 32 of dental model jig 30 that is within cavity 22 upon assembly, and thereby fixes dental model jig 30 in place along the anterior-posterior direction. In other words, the dental model jig 30 is restrained from moving back and forth along the anterior-posterior direction. The body 23 further includes a pair of lateral wing portions 25 extending transversely therefrom and along the anterior-posterior wall of body 23. The wing portions 25 include a plurality of spaced recesses 27, such that wing fingers 26 are configured to mate in corresponding recesses 17 of raised walls 15 of posterior portion 14, thereby providing an interlocking stepped edge profile or finger joint arrangement. The fixation screw disc 40 includes a plurality of equally spaced, short spokes 42 extending radially outwardly therefrom to support gripping of the disc 40 by a user when loosening or tightening the grub screw with respect to the dental model jig 30.

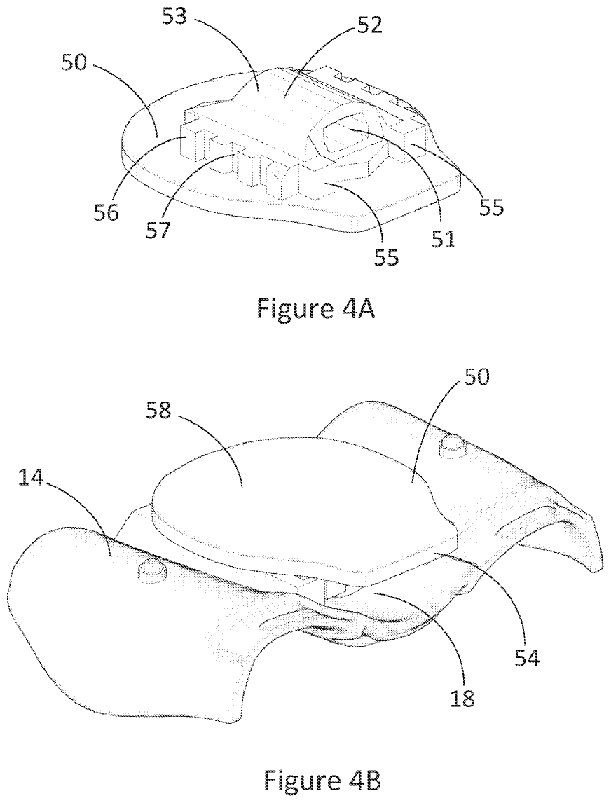

Reference is now made to FIGS. 4A and 4B, which illustrate the pin pad connector 52 and pin pad 50. The pin pad 50 and pin pad connector 52 may be separate components connected to one another in any suitable manner, or preferably provided by an integrally formed component. Pin pad connector 52 allows the pin pad 50 to be securely received by the coupling portion 18 of the posterior portion 14 in the same manner as dental model jig receiver 20. Pin pad connector 52 includes a body 53, which is of a generally similar shape as the body 23. The anterior end of the body 53 includes a cavity 51, through which a slider support body 32 (FIG. 5) of the dental model jig 30 can be received in an anterior-posterior direction. The body 53 further includes a pair of lateral wing portions 55 extending transversely therefrom and along the anterior-posterior wall of the body 53. The wing portions 55 include a plurality of alternating wing fingers 56 and recesses 57 configured to mate with corresponding fingers 16 and recesses 17 of raised walls 15 of posterior portion 14, thereby providing an interlocking stepped edge profile or finger joint arrangement. Pin pad 50 is configured to provide a bearing surface 58 for a vertical pin 86 (FIG. 8) that is used in the later described method in order to record a vertical dimension between the maxillary and mandibular. The periphery of the bearing surface of pin pad 50 is rounded as shown with an extended tapered anterior portion 54. In other embodiments, disc 40 may instead be used to provide a bearing surface for pin 86 in the later described method.

FIG. 5 illustrates the dental model jig 30 including the slider support body 32 in the general form of a rectangular prism with a convex lower facet as shown. The slider support body 32 is shaped and sized to closely fit within through cavity 22 for sliding along the anterior-posterior direction therewithin. Thus, the movement of the slider support body 32 allows for a positional adjustment of model teeth 36 in a first degree of freedom relative to posterior portion 14 when the dental model jig 30 has been received by the posterior portion 14 of maxillary impression tray 10. In the present embodiment, model teeth 36 take the form of a set of six teeth representative of a patient's six anterior maxillary teeth. However, in less preferred forms, the model teeth 36 need only to provide a representation of model teeth. In other words, the model teeth 36 need only to provide a positional or orientational relationship that can guide the design and/or manufacture of teeth for the desired dentures. The model teeth 36 need not necessarily mimic actual teeth, but instead be in the form of a bar or other component that can be used to mimic certain dimensions or orientations of two or more teeth.

It is preferred that the model teeth 36 are in the form of a set of six teeth representative of a patient's six anterior maxillary teeth because it allows a clinician to suitably position, in the anterior-posterior direction, the model teeth 36 and thereby ensure that the patient receives sufficient lip support by the six anterior maxillary teeth in the set of produced dentures.

Extending in an axial direction from the anterior-most wall of the slider support body 32 and connecting the slider support body 32 to the model teeth 36, is wire element 34. Wire element 34 is a plastically deformable wire, which through physical manipulation, allows for adjustment in a further five degrees of freedom to be provided between the model teeth 36 and the posterior portion 14 of maxillary impression tray 10. Thus, the apparatus 100 provides a total of six degrees of freedom of adjustment between the model teeth 36 and the posterior portion 14 of the maxillary impression tray 10, as illustrated in FIG. 5. As well as providing the multiple adjustments described above, the material of wire element 34 can withstand a degree of load without deforming, i.e. be retainable in the desired position. Hence, positioning and orientation of the model teeth 36 set during a procedure remains unchanged during the removal process of the model teeth 36 and upper portion 14, and during subsequent handling. The wire element 34 can be of any suitable form to achieve its function. For example, the wire element may be anodized aluminium, annealed copper, etc.

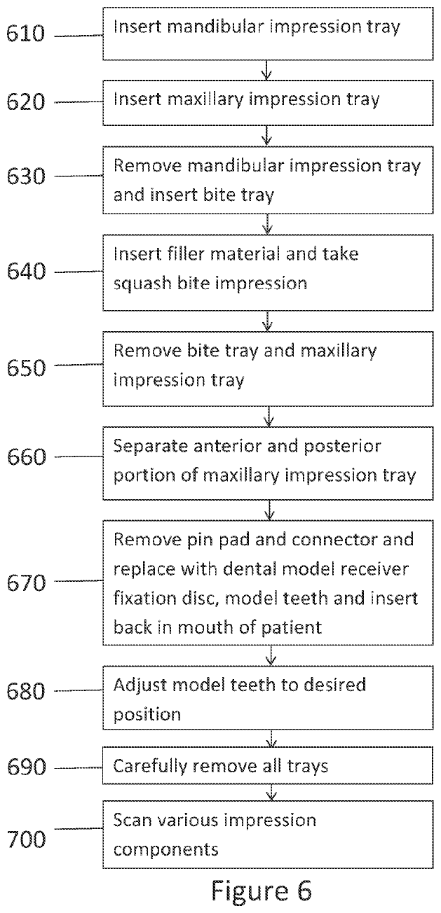

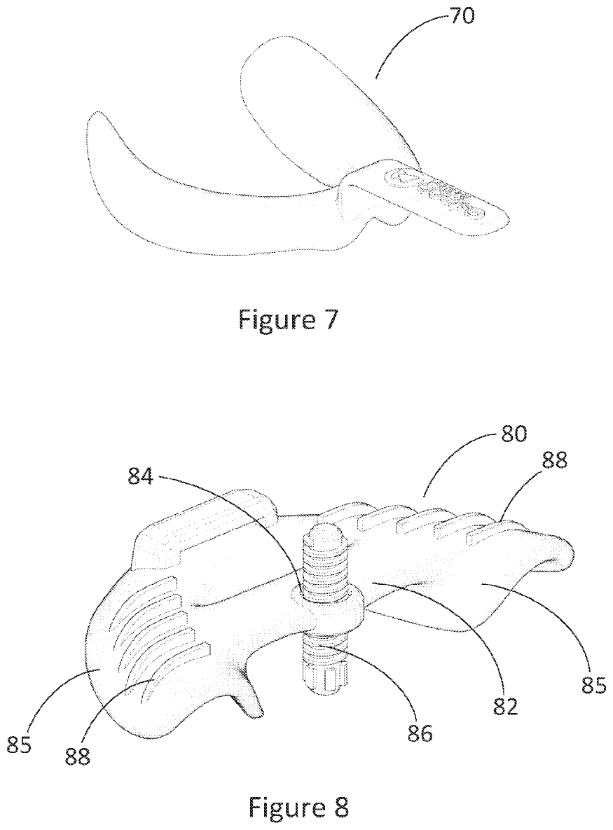

A method 600 according to an embodiment of the invention that utilises the apparatus 100 will now be described with reference to FIG. 6. In step 610, a mandibular impression tray 70 (FIG. 7), is provided with impression material and inserted into the mouth of the patient against the mandibular arch. In step 620, a maxillary impression tray 10, as previously described and shown in FIG. 2A, is provided with impression material and inserted into the mouth of the patient against the maxillary arch. At this stage, the anterior portion 12 and the posterior portion 14 are connected to form the full maxillary impression tray 10. The underside of the maxillary impression tray 10 has inserted therein the pin pad connector 52 and the pin pad 50.

In step 630, after the impression material has set, the mandibular impression tray 70 is carefully removed from the mouth of the patient and is replaced by a bite tray 80 (FIG. 8). In alternative embodiments, a standard maxillary impression tray may be initially used and then removed along with mandibular impression tray 70. In such an embodiment, the clinician would then insert maxillary impression tray 10, along with bite tray 80. The bite tray 80 is provided with impression material before insertion into the patient's mouth. The bite tray 80 is similar in overall shape to the mandibular impression tray 70, but much shorter in the posterior direction in order to ensure that the posterior ends of the maxillary impression tray 10 and the bite tray 80 do not clash. In another embodiment, the mandibular impression tray 70 may have removable posterior portions, which upon removal provide an impression tray in accordance with bite tray 80. The bite tray 80 further includes a bridging structure 82, which extends transversely between the posterior wing portions 85 of the bite tray 80. The bridging structure 82 includes a threaded aperture 84 for receiving the vertical threaded pin 86, which provides a screw whose end is intended to bear against the lower bearing surface 58 of pin pad 50. Bite tray 80 also includes a plurality of transversely extending locating ribs 88 protruding from the upper surface of posterior wing portions 85 as shown in FIG. 8. The function of the ribs 88 will be described later. Adjustment of pin 86 is used to assist setting and recording the desired vertical spacing between the bite tray 80 and the maxillary impression tray 10, i.e. the spacing between the upper and lower arch when the patient's mouth is closed. Pin 86 is adjusted by rotation of a knurled lower end until it bears on the bearing surface 58 of the pin pad 50 at the desired jaw spacing. The pin pad 50 thus provides a fixed reference point for the threaded vertical pin 86 with respect to the bite tray 80 and maxillary impression tray 10. In an alternative form, a separate bite tray 80 is not required, and the mandibular impression tray 70 is provided with detachable posterior portions that can be detached when a squash bite impression is to be taken. In such a configuration, the mandibular impression tray 70 also provides a pin in a suitable manner.

In step 640, a filler material is now inserted into the space between the bite tray 80 and the maxillary impression tray 10. This will allow the patient to provide a squash bite impression, with the squash bite impression defined in part by the spacing set by the vertical pin 86. The squash bite provides a full 3D impression of the spacing between the patient's mandibular and maxillary arches.

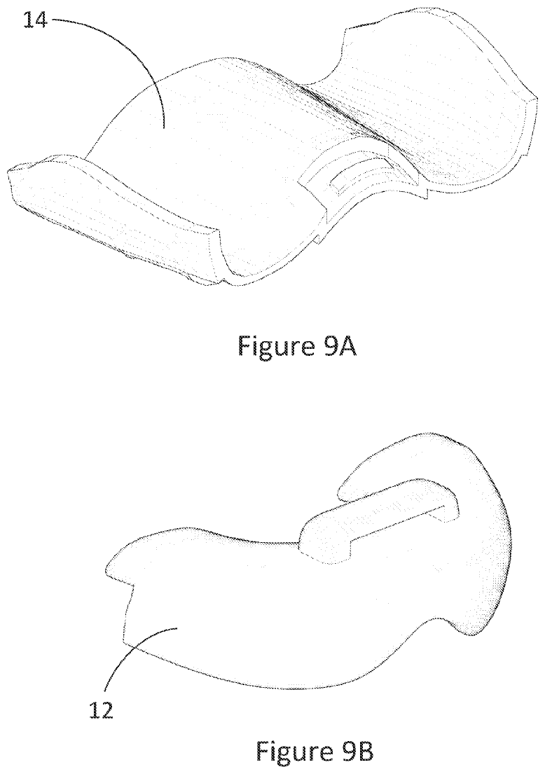

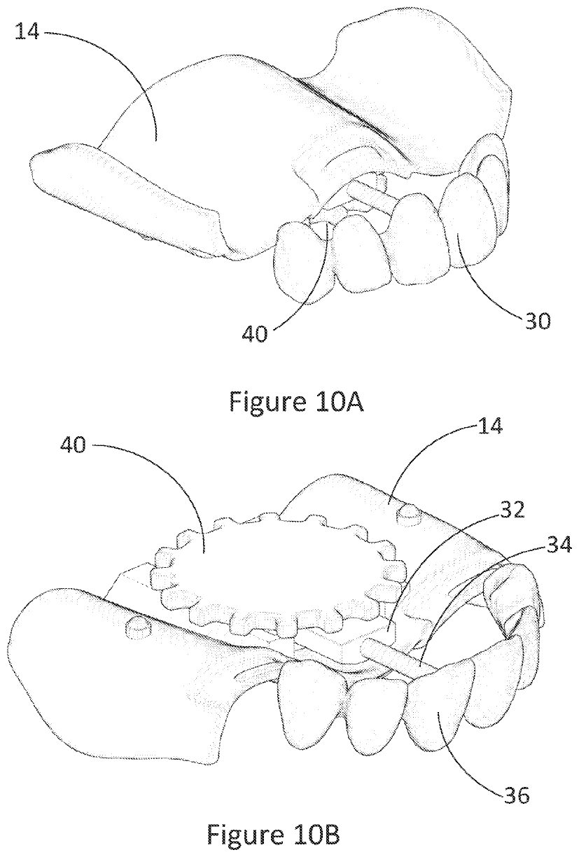

At step 650, the bite tray 80, the squash bite material, and the maxillary impression tray 10 are carefully removed from the patient's mouth. At step 660, the anterior portion 12 of the maxillary impression tray 10 is separated from the posterior portion 14. The separated posterior and anterior portions are shown in FIGS. 9A and 9B respectively. This separation can be achieved by cutting through the set impression material and along the boundary between the anterior portion 12 and posterior portion 14. This separation of the anterior portion 12 and posterior portion 14 allows for the dental model jig 30 to now be connected to the posterior portion as will be described. At step 670 the pin pad 50 and pin pad connector 52 are removed from the coupling portion 18 of the posterior portion 14. In their place, dental model receiver 20, fixation disc 40 and a suitable dental model jig 30 are inserted at coupling portion 18 as shown in FIGS. 10A and 10B. However, the clinician may choose to not make this substitution, and instead insert the slider support body 32 through cavity 51 of the pin pad connector 52, which then performs the same function as dental model jig receiver 20. This may be preferable if the clinician would like to keep the vertical pin 86 in place when establishing the position of model teeth 36. A suitable dental model 30 is selected based in part on the size of the maxillary impression tray 10 used in the method, e.g. small, medium, large and extra-large trays will each have a corresponding dental model jig 30. The dental model jig 30 thereby provides a suitably-sized representation of the model teeth 36 appropriate for the particular patient's mouth. The slider support body 32 of the dental model jig 30 is inserted through the cavity 22 of the dental model jig receiver 20. The posterior portion 14, with the dental model jig receiver 20, fixation screw disc 40 and dental model jig 30 are inserted back into the mouth of the patient at the same location as before.

At step 680, the model teeth 36 can be adjusted in six degrees of freedom. Preferably, the anterior-posterior adjustment is first realised by moving the slider support body 32 posteriorly and anteriorly until model teeth 36 is at the correct position, which includes providing the patient with sufficient lip support. Once this position has been determined, disc 40 is rotated so that its grub screw engages the slider support body 32 and prevents further anterior-posterior movement of the support body 32 and hence the model teeth 36. The model teeth 36 are then physically manipulated about any or all of the other five degrees of freedom until the desired position of the model teeth 36 is reached. From this desired positon, the clinician is able to accurately record information such as tissue impression of patient's mouth, jaw relationships, anterior tooth positions, midline, overbite, cant, anterior lean (inclination), and smile-line. The patient is also able to receive an indication as to how their smile will look when the completed dentures are provided.

At step 690, once the desired position of the model teeth 36 is located, the posterior portion 14 along with the components connected thereto are carefully removed from the patient's mouth, ensuring that the model teeth 36 are not moved from the set desired relative position and orientation.

At step 700, the following components are individually 3D scanned in 5-axis, capturing 360 degrees of the impressions: a) maxillary impression tray 10 with posterior portion 14 and anterior portion 12 connected to one another, b) posterior portion 14 of maxillary impression tray 10 with dental model jig 30 connected thereto, c) mandibular impression tray 70, d) bite tray 80, and e) an assembly of the bite tray 80, squash bite material, and the posterior portion 14 of the maxillary tray 10 (i.e. articulated impression). In the case of the scanned assembly of item e), the squash bite material will include grooves on its lower surface that were formed by the locating ribs 88 and notches formed on the squash bite material's upper surface that were formed by the positioning bosses 19, when the squash bite impression was taken. Thus, the locating ribs 88 and the positioning bosses 19 provide locating features in the squash bite material that allows for accurate assembly of the bite tray 80, squash bite material, and the posterior portion 14 of the maxillary tray 10 for the assembled scan of item e).

The scans are then utilised in appropriate dental software, in which the separate scans are merged into one global coordinate system to provide the full 3D data set to be used in the designing of the patient's dentures. In an embodiment of the described method, the clinician can in addition take a photograph of the assembled dental model jig 30 in the mouth of the patient with the model teeth 36 in the desired position. This photograph can then be overlayed onto the 3D model (in a single orientation only, i.e. the front view of the patient) and can guide parts of the design and/or provide a means to validate the subsequent design of the teeth. This is because the photograph can provide a reasonably accurate reflection of the patient's cant and midline. From these designs, a set of dentures can be manufactured. For example, the set of dentures may be manufactured utilising 5-axis milling machines, moulding, 3D printing, etc.

Thus, the use of the apparatus 100 allows (in a single visit to a dental clinic) no only the soft tissue geometries of the patient to be captured, but also the final teeth position. After these records are captured, they are then digitised via virtual denture design software which uses the impression system to guide the design of the dentures based on both the tissue and teeth positions. In particular, the use of a dental model jig 30 with a set of the six anterior model teeth 36 acts as a locator of the main “smile teeth” from which the remainder of the dentures can be built around. For this reason, there is no necessity to provide model teeth in the same way on the mandibular impression tray for the lower row of teeth. Setting the upper anterior set of teeth is generally sufficient to design a patient's occlusal as key parameters, such as midline, allow for the remaining teeth to be interpolated from the main six anterior model teeth 36.

The apparatus 100 and method 600 thereby allows the following measurements to be recorded from a single appointment with the patient: tissue impression of patient's mouth, jaw relationships, anterior tooth positions, midline, overbite, cant, anterior lean (inclination), and smile-line. From this, the dentures can be designed and manufactured accurately to suit the patient, thereby allowing fitting of the dentures to take place in a second and final appointment. This allows substantial reduction in costs for the patient, reduction in chair time for the patient, and can provide a more profitable and efficient solution for the clinician.

The CAD models that are produced from the scans provide a digital record of the various components of the dentures, which are then produced through the use of suitable CAM software that is used to program the operations for machining the components of the dentures.

Having a digital record of the dentures also allows for simpler post-processing, for example the manufacture of any components that may be damaged and require replacing. The component can be simply reproduced from its respective digital record. As will be appreciated by a person skilled in the art, any component of the dentures can be easily upgraded. For example, a more durable material can be utilised to produce a component that has previously failed during use.

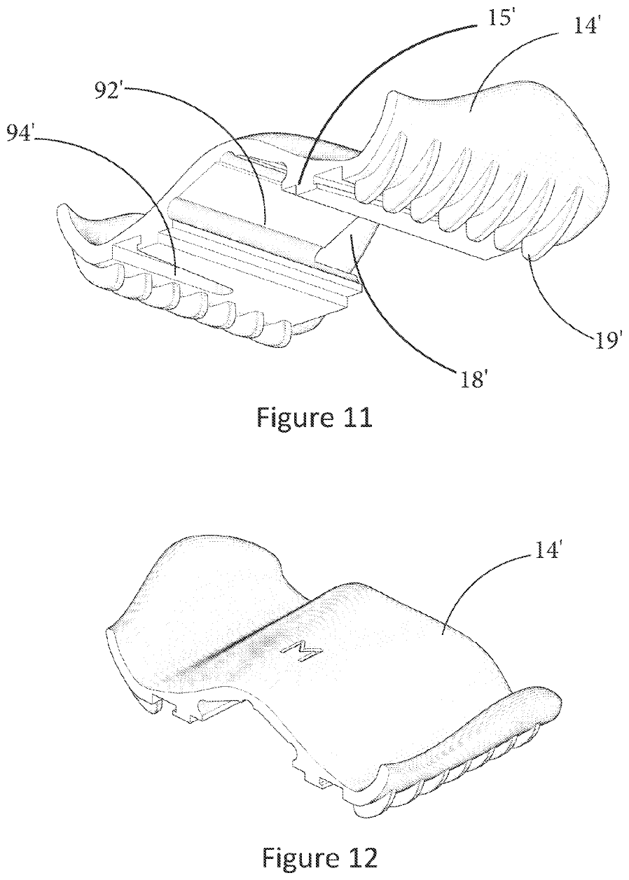

FIGS. 11-16 illustrate components of an apparatus of a second embodiment of the present invention. Components of this second embodiment are referenced using like reference numerals to those used with respect to the first embodiment (where applicable), with the addition of an apostrophe. It will appreciated that unless otherwise stated, the features shown in the second embodiment have the same function as the corresponding feature of the first embodiment. It will also be appreciated that the methodology involving the apparatus of the second embodiment is substantially the same as that involved using apparatus 100 of the first embodiment.

FIGS. 11 and 12 illustrate a posterior portion 14′ of a maxillary impression tray of the second embodiment. Posterior portion 14 includes a plurality of transversely extending locating ribs 19′ protruding from the lower surface of posterior portion 14, on either side of coupling portion 18′. Similar to the bosses 19 in the first embodiment, ribs 19′ will producing corresponding grooves in the squash bite material when the squash bite impression is taken. Thus, the locating ribs 19′ provide locating features in the squash bite material that allows for accurate assembly of the posterior portion 14′ for the assembled scan. Ribs 19′ also promotes flow of the bite material into desired areas while limiting the flow of bite material into other areas.

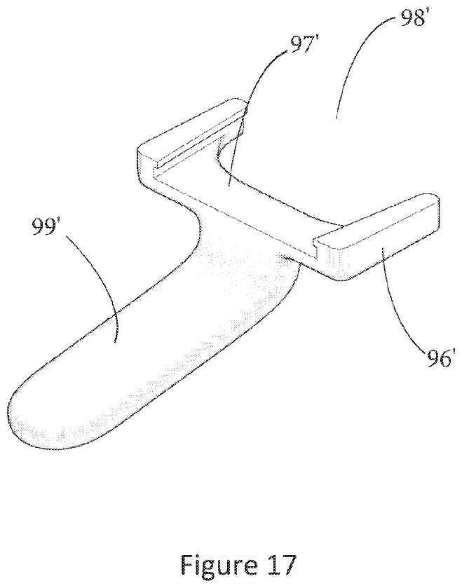

Coupling formation 18′ includes a pair of shaped walls 15′ protruding vertically downward from the lower surface of the posterior portion 14′ and extending longitudinally for most of the distance from the posterior end to the anterior end of posterior portion 14′, as shown. The two walls 15′ and the shaping of the surface of portion 14′ define an elongate channel 92′ therebetween, which in cross section has a substantially triangular shape with rounded vertices, as shown in FIGS. 11 and 12. Disposed between locating ribs 19′ and raised walls 15′ on each side of posterior portion 14′ is a handling groove 94′. The two handling grooves 94′ are configured to receive complementarily shaped gripping portions 96′ of a handling appliance 98′ (FIG. 17), used to facilitate handling of the posterior portion 14′, i.e. manoeuvring the posterior portion 14′ in and out of the mouth of a patient.

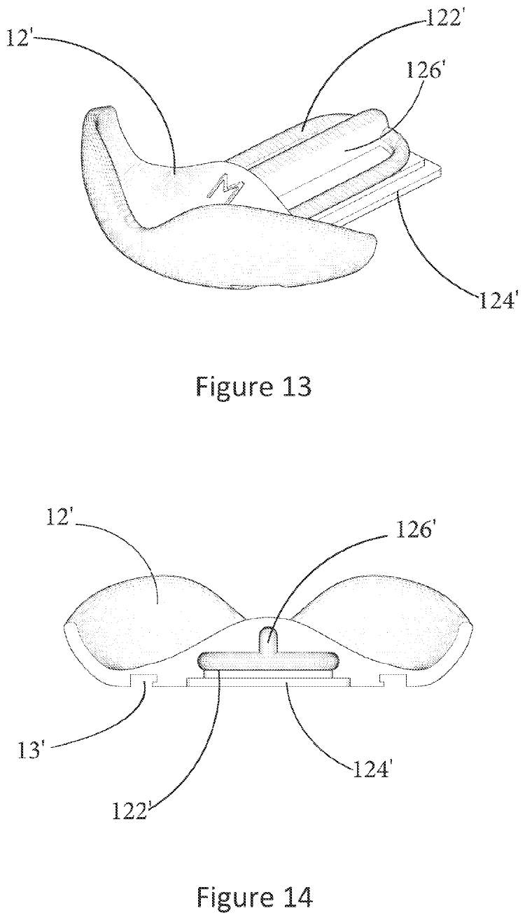

Reference is now made to FIGS. 13 and 14, which illustrates anterior portion 12′ of the maxillary impression tray of the second embodiment, configured to engage with posterior portion 14′ in order to form the full maxillary impression tray. Anterior portion 12′ includes an anterior-to-posterior extending tongue 122′ configured to engage with coupling formation 18′. Tongue 122′ includes a base portion 124′, having a stepped configuration, wherein a top portion thereof is recessed inwardly relative to a bottom portion of the base portion 124′. It will be appreciated that the shaped walls 15′ of posterior portion 14′ form complementary recesses for receiving base portion 124′ (as shown in FIG. 14). Tongue 122′ also comprises an upper retaining portion 126′ shaped to engage channel 92′ of posterior portion 14′. In particular, retaining portion 126′ has a generally elongate form with rounded upper and lateral parts. The shaping of these parts is complementary to the vertices of the cross section of channel 92′, enabling tongue 122′ to be received and matingly engaged with channel 92′ when the posterior portion 14′ is connected to anterior portion 12′. Anterior portion 12′ also includes handling grooves 13′ on either side, configured (like handling grooves 94′ of posterior portion 14′) to receive complementarily shaped gripping portions 96′ of handle 98′.

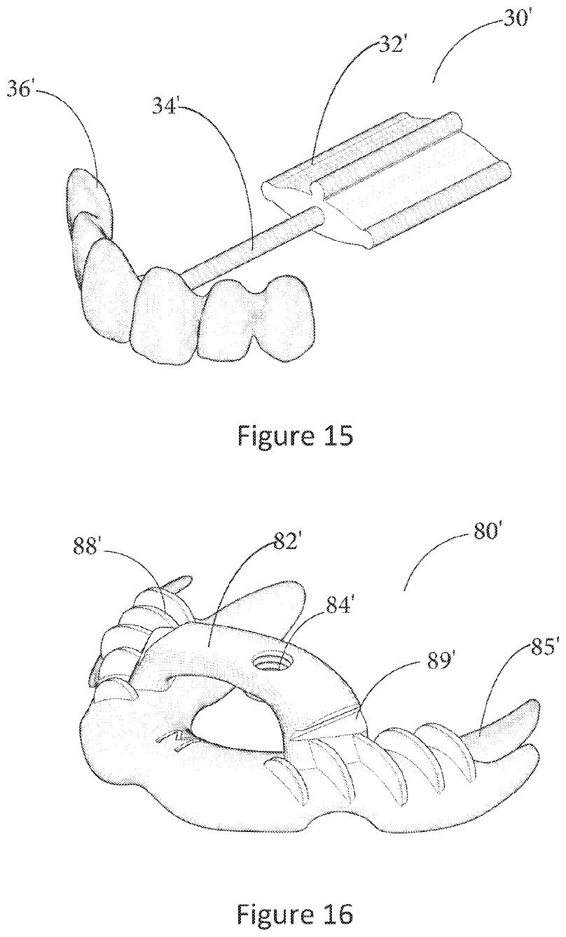

Reference is now made to FIG. 15, which illustrates a dental model jig 30′ configured to be engaged with posterior portion 14′. As will be appreciated from the drawings, this embodiment of the invention does away with the need for a separate and distinct component such as the dental model jig receiver 20 of the first embodiment. This is because sliding support body 32′ has a complementary shaping to that of coupling formation 18′ of posterior portion 14′, enabling the two to engage directly. To this end, sliding support body 32′ is in the general form of an elongate biconvex prism, which in section has opposed rounded lateral edges and a rounded elongate central formation on its upper side (as seen in FIG. 15) to matingly engage the rounded vertices of channel 92′ when the dental model jig 30′ is connected to posterior portion 14′. This form fit engagement allows dental model jig 30′ to be adjusted in the anterior-posterior direction by sliding along coupling formation 18′ in order to select the desired position.

Reference is now made to FIG. 16, which illustrates a bite tray 80′. Bite tray 80′ is similar in overall shape and configuration to bite tray 80 of the first embodiment. However, bite tray 80 includes a raised bridging structure 82′ between wing portions 85′, which includes handling grooves 89′ on either end of the bridging structure 82′ as shown. Similar to handling grooves 94′, handling grooves 89′ are configured to receive complementarily shaped gripping portions 96′ of handle 98′. Wing portions 85′ of bite tray 80′ also includes shaped posterior ends, which help to prevent any interference between the posterior portion 14′ and the bite tray 80′ when the bite tray 80′ in the patient's mouth. The shaped posterior ends also assist in spacing of the posterior tissue areas of the patient's mouth to allow visual inspection of the tray positioning.

FIG. 17 illustrates the handle 98′, which includes an elongate tongue-shaped handle portion 99′ and a head portion 97′. Head portion 97′ includes a pair of spaced lateral elongate gripping portions 96′, which serve the function of engaging with and gripping the complementary grooves in each of the trays as described above.

As previously mentioned, the methodology of using the apparatus of the second embodiment is substantially the same as that of the first embodiment, with any differences that arise due to the second embodiment readily apparent to the skilled reader.

It will be understood that the invention disclosed and defined in this specification extends to all alternative combinations of two or more of the individual features mentioned or evident from the text or drawings. All of these different combinations constitute various alternative aspects of the invention.

1. An apparatus for a denture preparation assembly, said apparatus configured for scanning to facilitate the digital design and/or manufacture of dentures for a patient, the apparatus comprising:
an impression tray piece for insertion into the mouth of the patient and to receive impression material; and
a dental model jig attachable to the impression tray piece, said dental model jig including model teeth;
wherein the attachment between the impression tray piece and the dental model jig is adjustable to allow the dental model jig to be moved in the mouth of the patient to a desired position relative to the impression tray piece, said relative position being retainable to allow scanning of the impression material and the dental model jig.

2. The apparatus of claim 1, wherein the attachment between the impression tray piece and the dental model jig is selectively lockable to fix the relative position therebetween.

3. The apparatus of claim 1, wherein the dental model jig is slideably attached to the impression tray piece to allow movement of the dental model jig in an anterior-posterior direction.

4. The apparatus of claim 3, further including a mechanical locking element to selectively block movement of the dental model jig with respect to the impression tray piece in the anterior-posterior direction.

5. The apparatus of claim 1, wherein the dental model jig further includes a connecting portion, wherein the connecting portion affords adjustment of the dental model jig in the mouth of the patient to a desired position relative to the impression tray piece.

6. The apparatus of claim 5, wherein the connecting portion is a plastically deformable material.

7. The apparatus of claim 6, wherein the connecting portion is a ductile wire.

8. The apparatus of claim 5, further including a support portion receivable by the impression tray piece and connected to the model teeth by the connecting portion.

9. The apparatus of claim 8, wherein the support portion includes a screw element engageable with a threaded element for locking the dental model jig in position relative to the impression tray piece.

10. The apparatus of claim 1, wherein the model teeth comprise a plurality of model teeth provided on the dental model jig to mimic real teeth in appearance.

11. The apparatus of claim 1, wherein the impression tray piece is part of a multi-part impression tray having a detachable anterior tray portion, detachment of the anterior tray portion allowing the attachment of the dental model jig to the impression tray piece.

12. A denture preparation kit including one or more of the following:
a maxillary impression tray;
a mandibular impression tray; and
an apparatus for a denture preparation assembly in accordance with claim 1, wherein the dental model jig can be chosen from a plurality of different sized dental model jigs in accordance with the size of the jaw.

13. A method for use in denture preparation for a patient, the method comprising:
loading an impression material into a first lower impression tray and inserting said lower impression tray into the patient's mouth to take a lower gum impression;
removing the first lower impression tray from the patient's mouth;
loading an impression material into a second upper impression tray and inserting said second upper impression tray into the patient's mouth to take an upper gum impression;
removing the second upper impression tray from the patient's mouth;
attaching a dental model jig to the second upper impression tray, said dental model jig including model teeth;
re-inserting the second upper impression tray into the patient's mouth over the upper gum;
adjusting the dental model jig relative to the second upper impression tray to a desired position,
removing the second upper impression tray and dental model jig.

14. The method of claim 13, further including, after the lower impression tray has been removed from the patient's mouth, loading an impression material into a second lower impression tray, the second lower impression tray including an upwardly projecting adjustable intra-jaw spacer, inserting the second lower impression tray into the patient's mouth, and adjusting the intra-jaw spacer.

15. The method of claim 13, further including, before the second upper impression tray has been inserted into the patient's mouth, loading an impression material into a first upper impression tray, inserting the first lower impression tray into the patient's mouth to take an upper gum impression, and removing the first upper impression tray.

16. The method of claim 13, wherein the first upper impression tray includes the second upper impression tray and a removable anterior tray portion, wherein when the first upper impression tray has been removed from the patient's mouth, the impression material is cut and the removable anterior tray portion removed from the second upper impression tray to allow attachment of the dental model jig, the impression tray then returned to the patient's mouth.

17. The method of claim 14, wherein the second upper impression tray includes a downward-facing bearing surface against which the intra-jaw spacer bears.

18. The method of claim 14, further including introducing a squash bite material between the second lower impression tray and the second upper impression tray and taking a squash bite impression, the squash bite impression defined in part by the spacing set by the intra-jaw spacer.

19. The method of claim 13, further including scanning the impression provided by the upper and lower impression trays and the attached dental model jig for use in digital design and/or manufacture of a denture set.

20. A method of denture manufacture, including receiving digital data provided by scanning the impressions provided by the upper and lower impression trays and the attached dental model jig resulting from carrying out the method of claim 13, and manufacturing a denture set in accordance with the digital data.
